(19) United States
(12) Plant Patent Application Publication
(10) Pub. No.: US 2002/0029397 P1
(43) Pub. Date: Mar. 7, 2002

(54) Verbena plant named 'Sunvivapa'

(76) Inventors: Yuki Watanabe, Kashiwa-shi (JP); Ryuichi Tachibana, Kawasaki-shi (JP)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 09/987,811

(22) Filed: Nov. 16, 2001

Related U.S. Application Data
(63) Continuation of application No. 09/375,408, filed on Aug. 17, 1999.

(51) Int. Cl. A01H 5/00

(52) U.S. Cl. PLT/308

(57) ABSTRACT

Disclosed herein is a Verbena plant which has erect growth habit and good plant height. The plant has plentiful flowers in a spike, and great profusion of blooms. The blooming period is late April to November and flowering duration is long. The whole bush remains in bloom for a considerable period of time. The flower size is large and the petal color of the flowers is vivid reddish purple without eye. The plant is highly tolerant to heat and cold, and has high resistances to pests and diseases, particularly powdery mildew, and high resistance to rain.

### BACKGROUND OF THE VARIETY

The present invention relates to a new and distinct variety of verbena plant obtained from crossing a Verbena hybrida ‘Showtime blaze’ (♀) and a wild type of verbena plant Verbena peruviana (♂) native to Brazil.

Verbena is a very popular plant and is used for flower bedding and potting in the summer season. There are only a few varieties of Verbena plants which is much branching, many flowers in a spike, a long flowering duration and which have a high-resistance to rain, heat, cold, and disease. Accordingly, this invention was aimed at obtaining a new variety having a erect growth habit, strong branching, many flowers in a spike, long flowering duration, high tolerance to heat and cold, and resistances to diseases and pests, and also having a vivid reddish purple color petal.

The new variety of Verbena plant according to this invention originated from crossing a Verbena hybrida ‘Showtime blaze’ (♀) and a wild type of verbena plant Verbena peruviana VBC (♂) native to Brazil.

First of all, 45 seedling were obtained in the autumn of 1992, from crossing of Verbena hybrida ‘Showtime blaze’ as female parent and a wild type of verbena plant VBC (Verbena peruviana f. rosea) as pollen parent in May of 1992. From this crossing, 12 seedlings were selected in view of erect growth habit,branching and petal color, and propagated by cutting, and then grown as a trial by flower bedding and planter from in the spring of 1993 to the autumn of 1994. The botanical characteristics of 12 seedlings were examined and only one seedling was selected, using parents varieties ‘Showtime blaze’ and a wild type of Verbena plant VBC for comparison. As a result, it was concluded that this selected Verbena is distinguishable from any other variety whose existence is known to us, and is uniform and stable in its characteristics. This new variety of Verbena plant was named ‘Sunvivapa’.

In the following description, the color-coding is in accordance with the Horticultural Color Chart of The Royal Horticultural Society, London, England (R.H.S. Color Chart), and the Inter-Society Color Council—National Bureau of Standards Color Name (I.S.C.C.—N.B.S. Color Name). A color chart based on The Japan Color Standard for Horticultural Plant (J.H.S. Color Chart) is also added for -reference.

The female parent ‘Showtime blaze’, used in the obtaining of this new variety ‘Sunvivapa’ is on the market. The main botanical characteristics of ‘Showtime braze’ are as follows.

Plant:

Growth habit.—Erect.

Plant extension.—Medium.

Plant height.—High. (25-35 cm).

Stem:

Diameter.—Medium.

Anthocyanin pigmentation.—Absent.

Pubescence.—Present.

Prickle.—Absent.

Branching.—Medium.

Subterranean stem.—Absent.

Length of internode.—Medium. (3-4 cm).

Leaf:

Phyllotaxis.—Opposite.

Shape of blade.—Hastate. (Type II).

Blade incision.—Present.

Depth of blade incision.—Shallow.

Shape of leaf margin.—Serrated.

Length.—Medium.

Width.—Medium.

Color.—Dark green. (R.H.S. color chart No. 137B, JHS color chart No. 3508).

Pubescence.—Present.

Petiole.—Absent.

Flower:

Shape of cluster.—Obconical. (Type II).

Spike length.—Medium.

Spike diameter.—Medium.

Facing direction.—Upward.

Outward curvature of petal.—Curved.

Diameter.—Large.

Height.—Long.

Color of petal.—Deep reddish orange (R.H.S. color chart No. 47A, J.H.S. color chart No. 0708).

Eye color.—Absent.

Varigation.—Absent.

Color presentation.—Substantially even.

Overlapping of petals.—Separate.

Incision of petal.—Present.

Number of petals.—Medium.

Calyx incision.—Present.

Calyx length.—Long.

Anthocyanin pigmentation of calyx limb.—Present.

Pistil shape.—Two lobes.

Stamen number.—Medium.

Anther color.—Yellowish green.

Peduncle diameter.—Thin.

Peduncle length.—Short.

Number of flowers.—Medium.

Flower fragrance.—Absent.

Flowering time.—Medium.

Flowering duration.—Medium.

Physiological and ecological characteristics:

Tolerance to cold.—Medium.

Tolerance to heat.—Low.

Resistance to diseases.—Low.

Resistance to pests.—Low.

The pollen parent a wild type of Verbena plant VBC was used in the obtaining of this new variety ‘Sunvivapa’ is a wild type of verbena native to South Brazil and Verbena peruviana. This wild type of verbena plant is presently maintained at the Hakushu Nursery Center of SUNTORY Ltd., residing at 2913-1 Torihara, Hakushu-cho, Kitakoma-gun, Yamanashi-ken, Japan. The main botanical characteristics of this pollen parent VBC are as follows.

Plant:

Growth habit.—Erect.

Plant extension.—Medium.

Plant height.—High.

Stem:

Diameter.—Medium. (2-3. mm).

Anthocyanin pigmentation.—Present.

Pubescence.—Dense.

Prickle.—Absent.

Branching.—Abundant.

Subterranean stem.—Absent.

Length of internode.—Medium.

Leaf:

Phyllotaxis.—Opposite.

Shape of blade.—Hastate. (Type II).

Blade incision.—Present.

Depth of blade incision.—Shallow.

Shape of leaf margin.—Crenated.

Length.—Medium.

Width.—Medium.

Color.—Dark green. (R.H.S. color chart No. 137B, JHS color chart No. 3508)

Pubescence.—Dense.

Petiole.—Absent.

Flower:

Shape of cluster.—Turbinate. (Type I).

Spike length.—Medium.

Spike diameter.—Medium.

Facing direction.—Upward.

Outward curvature of petal.—Curved.

Diameter.—Medium.

Height.—Medium.

Color of petal.—Brilliant purple (R.H.S. color chart No. 86D, J.H.S. color chart No. 8305).

Eye color.—Present.

Size of eye color.—Medium.

Varigation.—Absent.

Color presentation.—Substantially even.

Overlapping of petals.—Separate.

Incision of petal.—Present.

Number of petals.—Medium.

Calyx incision.—Present.

Calyx length.—Medium.

Anthocyanin pigmentation of calyx limb.—Present.

Pistil shape.—Two lobes.

Stamen number.—Medium.

Anther color.—Yellowish green.

Peduncle diameter.—Medium.

Peduncle length.—Medium.

Number of flowers.—Medium.

Flower fragrance.—Absent.

Flowering time.—Very late.

Flowering duration.—Long.

Physiological and ecological characteristics:

Tolerance to cold.—High.

Tolerance to heat.—High.

Resistance to diseases.—High.

Resistance to pests.—High.

The new variety of Verbena plant ‘Sunvivapa’ was asexually reproduced by cutting at the aforementioned Hakushu Nursery Center of SUNTORY Ltd., residing at 2913-1 Torihara, Hakushu-cho, Kitakoma-gun, Yamanashi-ken, Japan, and the homogeneity and stability thereof were confirmed.

### SUMMARY OF THE VARIETY

This new variety of Verbena plant has erect growth habit and plant height is high. The plant has many branchings and plentiful flowers in a spike, and great profusion of blooms. The blooming period is late April to November and the flowering duration is long. The whole bush remains in bloom for a considerable period of time. The flower petal color is vivid reddish purple color and have not eye. The plant is highly tolerant to heat, cold, and has a high resistance to pests and diseases, particularly powdery mildew, and high resistance to rain.

### BRIEF DESCRIPTION OF THE DRAWINGS

FIG. 1 is photograph giving a partial view of the new variety of verbena plant planted in the ground.;

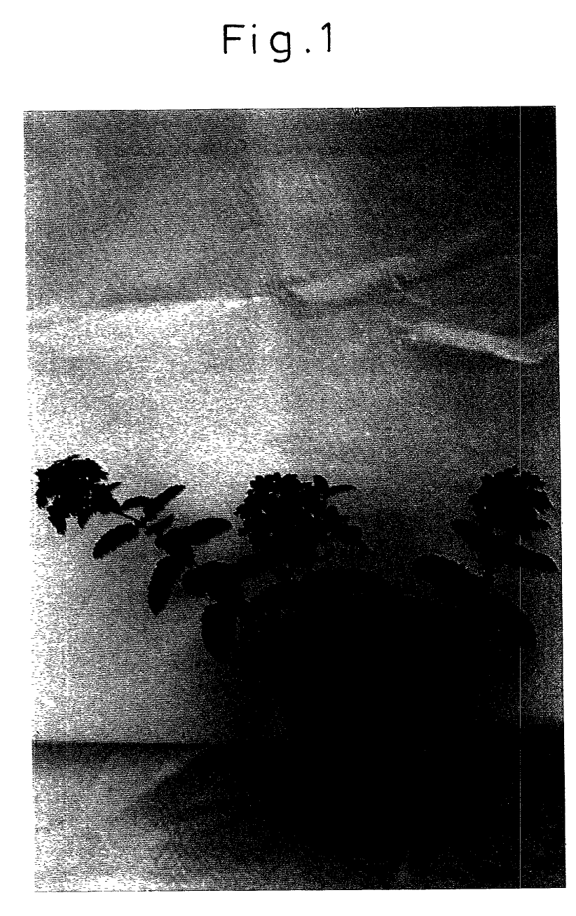

FIG. 2 is a photograph of flowers of the new variety of verbena plant.

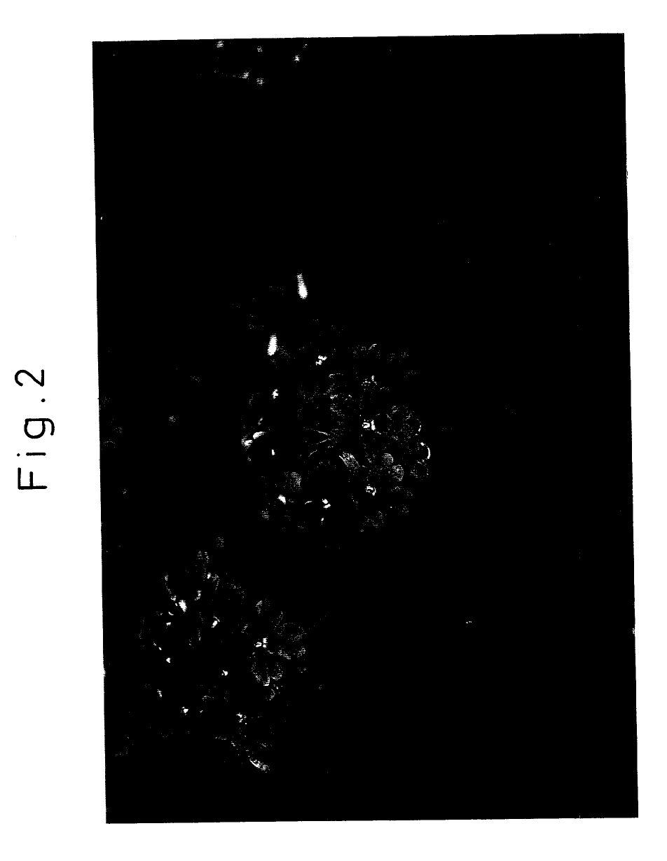

### DESCRIPTION OF THE VARIETY

The botanical characteristics of the new and distinct variety of verbena plant, ‘Sunvivapa’ are as follows.

Plant:

Growth habit.—Erect.

Plant extension.—Medium.

Plant height.—High. (40-50 cm).

Stem:

Diameter.—Medium. (2-3. mm).

Anthocyanin pigmentation.—Present.

Pubescence.—Dense.

Prickle.—Absent.

Branching.—Abundant.

Subterranean stem.—Absent.

Length of internode.—Medium. (4-5 cm).

Leaf:

Phyllotaxis.—Opposite.

Shape of blade.—Hastate. (Type II).

Blade incision.—Present.

Depth of blade incision.—Shallow.

Shape of leaf margin.—Crenated.

Length.—Medium. (4-5 cm).

Width.—Medium. (3 cm).

Color.—Moderate olive green. (R.H.S. color chart No. 146A, J.H.S. color chart No. 3509).

Pubescence.—Present.

Petiole.—Present.

Petiole diameter.—Medium.

Petiole length.—Medium.

Flower:

Shape of cluster.—Funnel-shaped (Type III).

Spike length.—Medium. (3 cm).

Spike diameter.—Large. (4-5 cm).

Facing direction.—Upward.

Outward curvature of petal.—Slightly curved.

Diameter.—Large. (20-30 mm).

Height.—Long.

Color of petal.—Vivid reddish purple (R.H.S. color chart No. 74A, J.H.S. color chart No. 8907).

Eye color.—Absent.

Varigation.—Absent.

Color presentation.—Substantially even.

Overlapping of petals.—Separate.

Incision of petal.—Present.

Number of petals.—Medium.

Calyx incision.—Present.

Calyx length.—Medium.

Anthocyanin pigmentation of calyx limb.—Present.

Pistil shape.—Two lobes.

Stamen number.—Medium.

Anther color.—Yellowish green.

Peduncle diameter.—Medium.

Peduncle length.—Medium.

Number of flowers.—Medium.

Flower fragrance.—Absent.

Flowering time.—Late.

Flowering duration.—Long.

Physiological and ecological characteristics:

Tolerance to cold.—High.

Tolerance to heat.—High.

Resistance to diseases.—High.

Resistance to pests.—High.

This new variety of Verbena plant is most suitable for flower bedding and potting, particularly in planters, and is further excellent for ground cover.

The plant of this new variety, ‘Sunvivapa’ is presently planted and maintained at the Hakushu Nursery Center of SUNTORY Ltd., residing at 2913-1 Torihara, Hakushu-cho, Kitakoma-gun, Yamanashi-ken, Japan.

1. A new and distinct variety of Verbena plant, substantially as herein illustrated and described, characterized particularly as to novelty by (A) having a erect growth habit, (B) having plentiful flowers in a spike and great profusion blooms, the whole bush remaining in bloom for a considerable period of time, (C) having a long flowering duration, (D) being the petal color of the flowers a vivid reddish purple without eye, (E) having high resistances to rain, heat, cold, drought, diseases and pests.
